(19) United States
(12) Patent Application Publication
(10) Pub. No.: US 2014/0271619 A1
(43) Pub. Date: Sep. 18, 2014

(54) CD20 CONFORMATIONAL ISOMERS AND METHODS OF USING

(71) Applicant: Strategic Diagnostics Inc., Newark, DE (US)

(72) Inventor: Ross Stewart Chambers, Hockessin, DE (US)

(21) Appl. No.: 13/794,921

(22) Filed: Mar. 12, 2013

(51) Int. Cl. C07K 14/705; A61K 39/395; G01N 33/68

(52) U.S. Cl. CPC C07K 14/70596; G01N 33/6893; A61K 39/3955
USPC 424/133.1; 536/23.5; 435/320.1; 435/369; 435/7.24; 424/139.1; 530/387.9

(57) ABSTRACT

Described herein are methods of screening for compounds that bind to conformational isomers of CD20 polypeptides. Also described are antibodies that bind to conformational isomers of CD20 polypeptides.

### TECHNICAL FIELD

This disclosure generally relates to CD20 polypeptides and antibodies that bind to CD20 polypeptides.

### BACKGROUND

CD20 is a non-glycosylated integral membrane protein of B cells that has four membrane spanning domains and two small loops on the surface. Human CD20 (encoded by a gene designated as MS4A1) is composed of 297 amino acids (aa) and migrates as a 33-37 kDa protein in SDS-PAGE, depending on its level of phosphorylation. CD20 is a calcium channel that replenishes internal calcium stores and maintains cytoplasmic calcium levels for sustained signal transduction after B cell activation. CD20 also is a constitutive component of lipid rafts, which are important for signaling and protein trafficking. Lipid rafts are membrane structures that are 10-200 nm in diameter and enriched in cholesterol, glycosphingolipids and GPI-anchored proteins.

### SUMMARY

Methods of screening for compounds that bind to conformational isomers of CD20 polypeptides are described herein. Also described are antibodies that bind to conformational isomers of CD20 polypeptides.

In one aspect, a mutant CD20 nucleic acid is provided. Such a mutant CD20 nucleic acid can have a sequence such as SEQ ID NO:2, SEQ ID NO:3, SEQ ID NO:4, or SEQ ID NO:5. A vector comprising such a mutant CD20 nucleic acid also is provided, as is a host cell comprising such a vector.

In another aspect, a method of screening for compounds that specifically bind to a conformational isomer of the CD20 polypeptide is provided. Such a method typically includes contacting a mutant CD20 polypeptide with a test compound, and determining whether or not the test compound binds to the mutant CD20 polypeptide. Generally, binding of the test compound to the mutant CD20 polypeptide indicates a compound that binds to a conformational isomer of the CD20 polypeptide.

In another aspect, a method of screening for compounds that specifically bind to a conformational isomer of the CD20 polypeptide and to a wild type CD20 polypeptide is provided. Such a method typically includes contacting a mutant CD20 polypeptide with a test compound; contacting a wild type CD20 polypeptide with the test compound; and determining whether or not the test compound binds to both the mutant CD20 polypeptide and the wild type CD20 polypeptide. Generally, binding of the test compound to both the mutant CD20 polypeptide and the wild type CD20 polypeptide indicates a compound that binds to both a conformational isomer of the CD20 polypeptide and to the wild type CD20 polypeptide.

In still another aspect, a method of screening for compounds that specifically bind to a conformational isomer of the CD20 polypeptide and induce a conformational change is provided. Such a method typically includes contacting a mutant CD20 polypeptide with a test compound in the presence of an extracellular epitope-binding antibody; and determining whether or not the antibody binds to the mutant CD20 polypeptide in the presence of the test compound. Generally, binding of the antibody to the mutant CD20 polypeptide in the presence of the test compound is indicative of a test compound that induces a conformational change in the conformational isomer of the CD20 polypeptide.

In some embodiments, the mutant CD20 polypeptide has a sequence selected from the group consisting of SEQ ID NO:2 (CD20+0) and SEQ ID NO:3 (CD20+6). In some embodiments, the mutant CD20 polypeptide contains a mutation in the cholesterol binding motif (GIVENEWKRTCS; SEQ ID NO:6). In some embodiments, the mutant CD20 polypeptide contains a cholesterol binding motif (GIVENEWKRTCS; SEQ ID NO:6) that is in a different position in the mutant CD20 polypeptide compared to a wild type CD20 polypeptide. Representative compounds include, without limitation, small molecules, polypeptides, synthetic compounds, naturally-occurring compounds, antibodies, antigen-binding fragments, or antigens. In some embodiments, the determining step utilizes FACS analysis. In some embodiments, the conformational isomer of the CD20 polypeptide is a CD20 polypeptide that is not specifically bound by an extracellular epitope-binding antibody.

In yet another aspect, a method of making an antibody that specifically binds a conformational isomer of the CD20 polypeptide is provided. Such a method typically includes immunizing a host animal with a mutant CD20 polypeptide. Also provided is an antibody directed toward a conformational isomer of a CD20 polypeptide, wherein the antibody is made by the method described herein.

In yet another aspect, a method of treating an individual suffering from an autoimmune disease is provided. Such a method typically includes identifying the individual as Rituxumab-resistant or Rituxumab-sensitive, wherein the individual is identified as Rituxumab-resistant if an antibody as described herein binds more polypeptides in a biological sample from the individual relative to Rituxumab or wherein the individual is identified as Rituxumab-sensitive if an extracellular epitope-binding antibody binds more polypeptides in a biological sample from the individual relative to an antibody as described herein; and administering Rituxumab to the individual if the individual is identified as Rituxumab-sensitive and administering an antibody as described herein to the individual if the individual is identified as Rituxumab-resistant.

In yet another aspect, a method of treating an individual who has an autoimmune disease but is not responding or is responding poorly to treatment with Rituxumab is provided. Such a method typically provides administering an effective amount of an antibody as described herein to the individual. Such a method can further include continuing to administer Rituxumab to the individual.

In still another aspect, a method of determining whether an individual diagnosed with an autoimmune disease will respond well or will respond poorly to treatment with Rituxumab is provided. Such a method typically provides evaluating the binding of an antibody as described herein to CD20 polypeptides in a biological sample from the individual. Generally, a high level of binding of an antibody as described herein to CD20 polypeptides in the biological sample indicates that the individual will respond poorly to treatment with Rituxumab, and wherein low levels of binding of an antibody as described herein to CD20 polypeptides in the biological sample indicates that the individual will respond well to treatment with Rituxumab. Representative autoimmune diseases include, for example, non-Hodgkin's lymphomas (NHL), Hodgkin's lymphoma, and rheumatoid arthritis (RA).

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a schematic showing the cloning strategy used to create the CD20 mutants described herein.

FIG. 2 shows the level of expression and binding, based on flow cytometry using either an anti-HA antibody (top) or an anti-CD20 antibody (bottom), of wild type CD20 (left) and the CD20+0 mutant (right) in the Snorkel construct in HEK293 cells.

FIG. 3 shows the level of expression and binding, based on flow cytometry using an anti-HA antibody and a number of different anti-CD20 antibodies, of wild type CD20 (top) and the CD20+0 mutant (bottom) in the Snorkel construct in HEK293 cells.

FIG. 4 shows the level of expression and binding, based on flow cytometry using an anti-HA antibody (top) and an anti-CD20 antibody (bottom), of wild type CD20 (SEQ ID NO:1) and each of the CD20 mutants described herein (CD20+0 (SEQ ID NO:2), CD20+6 (SEQ ID NO:3), CD20+12 (SEQ ID NO:4), and CD20+18 (SEQ ID NO:5)).

FIG. 5 shows the level of expression and binding, based on flow cytometry using a polyclonal mouse antiserum generated using wild type CD20 polypeptides, of wild type CD20 and each of the CD20 mutants described herein.

FIG. 6 shows CD20 sequences interrupted by dU linker sequences.

FIG. 7 are tables showing that the C-terminal domain juxtamembrane region (left; SEQ ID NOs:11-21) and the cholesterol recognition/interaction consensus sequence (right; SEQ ID NOs:22-41) in CD20 is conserved across orthologs.

FIG. 8 is an alignment of the CD20 extracellular domain juxtamembrane from a number of species (SEQ ID NOs:42-51).

Like reference symbols in the various drawings indicate like elements.

### DETAILED DESCRIPTION

CD20 is considered an ideal target for therapeutic antibodies because it is highly expressed (˜200,000 copies per cell); its expression is restricted to B cells, excluding lymphoid stem cells; it remains on the surface of cells even after antibody binding, thereby facilitating effecter function; and it is not shed and, thus, does not compete with antibody binding to the cells. Therefore, CD20 is one of the best validated drug targets for monoclonal antibody therapy and is the target of several approved therapeutic antibodies, most notably Rituxumab (RITUXAN™) for the treatment of B cell Non-Hodgkin's Lymphomas (NHL). However, current therapeutic antibodies have relatively poor efficacy and a high incidence of relapse. For example, Rituxumab is limited in that it is only effective in ˜50% of patients (i.e., ˜50% of patients are considered to be Rituxumab-resistant). While in some cases, resistance to Rituxumab appears to be due to a decrease in expression of CD20, other potential resistance mechanisms have been proposed and include mutation of the CD20 epitope, mutations in signaling pathways, the location of the B cells, and the level of tumor burden. With respect to mutations within CD20, however, DNA sequencing of the extracellular regions in NHL patients, either before or after Rituxumab treatment, showed that mutations in the extracellular region were rare.

As described herein, CD20 sequences were produced that were expressed well at the cell surface but were unable to bind CD20 antibodies directed to an extracellular epitope (e.g., the 2H7 antibody, the B1 antibody, or Rituxumab). The CD20 sequences described herein contained an insertion in or near the cholesterol binding motif, which prevented cholesterol binding. Thus, modifying the cholesterol binding motif in the CD20 polypeptides converted the polypeptide into a non-cholesterol binding conformation, which also did not bind to an extracellular epitope-binding antibody. Accordingly, these results suggest that CD20 exists in multiple conformations in vivo (i.e., CD20 conformational isomers), only some of which are able to be bound by an extracellular epitope-binding antibody. In addition, these results suggest that at least some of the CD20 conformational isomers are stabilized by changes in the cholesterol binding motif. Significantly, this means that, in clinical samples, there is the potential for a population of CD20 polypeptides that are unable to bind Rituxumab (i.e., Rituxumab-resistant) and, additionally, are not able to be detected in flow cytometry using CD20 antibodies similar to Rituxumab that also are directed toward the extracellular epitope.

Such conformational isomers of CD20 can be used to screen for compounds that bind to that isomer or to screen for compounds that act as allosteric modulators. In addition, antibodies can be generated to CD20 conformational isomers by immunizing animals with, for example, one or more CD20 sequences that adopt an alternate conformation. Such antibodies can be useful in the treatment of Rituxumab-resistant B cells for various indications including lymphomas, and also can be used as a biomarker to help guide treatment.

### Screening Methods

As described herein, a number of different CD20 sequences were generated that appear to represent conformational isomers of the wild type CD20 polypeptide. Based on the data presented herein, wild type CD20 polypeptides bind well to therapeutic antibodies such as Rituxumab and publicly available antibodies such as 2H7 and B1, and such binding results in antibody-dependent cellular cytotoxicity (ADCC). However, the CD20 conformational isomers, simulated by, for example, a “mutant CD20 sequences”, do not bind such antibodies and do not result in ADCC. Thus, mutant CD20 sequences such as those described herein can be used to screen for compounds such as antibodies or small molecules that bind, for example, to a conformational isomer of the CD20 polypeptide or that bind, for example, to both a conformational isomer of the CD20 polypeptide and to a wild type CD20 polypeptide.

For example, in some embodiments, a conformational isomer or both a conformational isomer and a wild type CD20 polypeptide can be contacted with a test compound, and routine methods then can be used to determine whether or not the test compound binds to the conformational isomer and/or the wild type CD20 polypeptide. As described herein, binding of a test compound to a CD20 conformational isomer indicates that the test compound binds a conformational isomer of the CD20 polypeptide, while binding of a test compound to both a conformational isomer and a wild type CD20 polypeptide indicates that the test compound is a pan-specific compound that is able to bind to both a conformational isomer of the CD20 polypeptide and the wild type CD20 polypeptide.

In addition, CD20 sequences such as those described herein can be used to screen for compounds that bind to a conformational isomer of the CD20 polypeptide and induce a change to the wild type CD20 conformation. For example, a CD20 conformational isomer (e.g., one that does not effectively bind to one or more extracellular epitope-binding antibody (e.g., 2H7, B1, or Rituxumab)) can be contacted with a test compound in the presence of an extracellular epitope-binding antibody, and routine methods then can be used to determine whether or not effective binding occurs. It would be understood by those in the art that binding of an extracellular epitope-binding antibody to a CD20 conformational isomer in the presence of the test compound identifies a test compound as one that is able to induce a change in the conformational isomer of the CD20 polypeptide such that CD20 polypeptides adopt the wild type conformation and are able to bind such an extracellular epitope-binding antibody.

It would be understood by those skilled in the art that, since CD20 is a transmembrane protein, the methods described herein can take place in vitro using membrane preparations. In vitro membrane preparations allow CD20 to assume its proper positioning and conformation in the cell membrane. Methods of obtaining membrane preparations are well known to those in the art. See, for example, U.S. Pat. Nos. 4,151,053 and 4,385,148, and Lee et al. (2008, Protein Expression and Purification, 62:223-9). In addition, it would be understood by those skilled in the art that the methods described herein also can take place in vivo using cells (e.g., whole, intact cells). Cells that can be used to evaluate binding of a compound to a wild type CD20 polypeptide or a CD20 sequence that adopts a conformation other than wild type include, for example, CHO cells, HEK293 cells, and NIH 3T3 cells, each of which are publicly available.

As discussed in the Example section, the CD20 conformational isomers described herein were generated by inserting a short 9 amino acid sequence into the region containing the cholesterol binding motif, thereby disrupting the motif. In addition to disrupting the motif by sequence insertion, CD20 conformational isomers also can be generated by moving the cholesterol-binding motif located on the intracellular side of the CD20 polypeptide away from the membrane (i.e., to a different position compared to the position of the cholesterol-binding site in the wild type CD20 polypeptide). Additionally, CD20 conformational isomers can be generated by introducing a mutation (e.g., a substitution or a deletion) into the cholesterol-binding motif (SEQ ID NO:6). Thus, as used herein, a “CD20 conformational isomer” refers to a CD20 polypeptide for which an extracellular epitope-binding antibody such as 2H7, B1 or Rituxumab exhibits altered binding affinity due to a change in the structure of the CD20 polypeptide. As described herein, CD20 conformational isomers can be generated by mutating CD20 polypeptides, where the mutation is in the position of the cholesterol-binding motif, in the sequence of the cholesterol-binding motif, or both.

It would be understood by those in the art that the methods described herein are not limited to using only the specific sequences disclosed herein. However, it also would be understood by those in the art that the CD20 sequences used in the methods described herein needs to adopt a conformational change. One criterion that can be used to determine whether or not a CD20 polypeptide sequence adopts a different conformation than wild type CD20 polypeptides is its antibody binding profile. As used herein, a wild type CD20 polypeptide is one that has a conformation that is recognized (i.e., specifically bound) by an antibody as discussed above that binds to the extracellular epitope such as, without limitation, the B1 antibody, the 2H7 antibody, or the Rituxumab antibody; a CD20 polypeptide has a non-wild type conformation if the CD20 polypeptide is not recognized by an antibody that binds to the extracellular epitope (e.g., the B1 antibody, the 2H7 antibody, or Rituxumab).

While flow cytometry can be used in these comparisons, other methods such as epitope tagging or use of an antibody that recognizes an internal epitope on the CD20 polypeptide (usually coupled with permeabilization of the cells) may be useful, for example, to detect the presence of internal pools of CD20 or to discriminate between surface and internal pools of CD20. As used herein, an antibody “specifically recognizes” or “specifically binds” to an antigen when a signal is produced in an immunoassay that is significantly greater (e.g., at least 2-fold, 5-fold, 10-fold, 30-fold, or 100-fold greater) than the signal produced by binding of the antibody to a control (e.g., a sample lacking the antigen).

There are a number of CD20 mutants described in the literature ((Δ1-49, Δ226-252, Δ230-245, Δ253-297) that appear to have wild type ANPS epitopes as defined by 2H7 antibody recognition in flow cytometry (Polyak et al., 1998, J. Immunol., 161:3242-48). Therefore, these mutants do not appear to adopt an altered conformation. There is also a CD20 mutant described in the literature (Δ219-225) that no longer associates with lipid rafts, and, based on antibody binding, may, at least partially, adopt a conformational change (Polyak et al., 1998, J. Immunol., 161:3242-48; Li et al., 2003, J. Biol. Chem., 278:42427). In addition, there are a number of CD20 mutations that have been identified in patients that exhibit resistance to treatment with Rituxumab, and include, for example, I211S, T219A with deletion after residue 230, E215G with deletion after residue 230, and deletion after residue 230 (see, for example, Terui et al., 2009, Clin. Cancer Res., 15:2523-30). These mutations identified in Rituxumab-resistant patients need to be evaluated further for their conformation (i.e., wild type or isomer), but represent additional CD20 sequences that can potentially be used in the methods described herein.

The polypeptides used in the methods described herein can be produced by any number of methods well known in the art. By way of example and without limitation, a polypeptide can be obtained by expression of a recombinant nucleic acid encoding the polypeptide or by chemical synthesis (e.g., by solid-phase synthesis or other methods well known in the art, including synthesis with an ABI peptide synthesizer; Applied Biosystems, Foster City, Calif.). In some cases, expression vectors that encode polypeptides provided herein can be used to produce a polypeptide. For example, standard recombinant technology using expression vectors encoding a polypeptide provided herein can be used. Expression vectors that can be used for small or large-scale production of a polypeptide include, without limitation, bacteriophage DNA, plasmid DNA, or cosmid DNA expression vectors containing the nucleic acid encoding the polypeptide.

The resulting polypeptides can be purified. In some cases, suitable methods for purifying the polypeptides of the invention can include, for example, immunoprecipitation, size exclusion chromatography, and ion exchange chromatography. The extent of purification can be measured by any appropriate method, including but not limited to: column chromatography, polyacrylamide gel electrophoresis, or high-performance liquid chromatography. A polypeptide can be designed or engineered to contain a tag sequence that allows the polypeptide to be purified (e.g., captured onto an affinity matrix). For example, a tag such as c-myc, hemagglutinin, polyhistidine, or FLAG™ tag (Kodak) can be used to aid polypeptide purification.

As indicated herein, the polypeptides used in the methods disclosed herein can be encoded by a nucleic acid. The term “nucleic acid” as used herein encompasses both RNA and DNA, including cDNA, genomic DNA, and synthetic DNA (e.g., chemically synthesized). The nucleic acid can be circular or linear, and can be double-stranded or single-stranded. Where single-stranded, the nucleic acid can be the sense strand or the antisense strand. Nucleic acids can be obtained using any appropriate method including, without limitation, common molecular cloning and chemical nucleic acid synthesis techniques. In some embodiments, nucleic acid molecules can be obtained by recombinant nucleic acid technology. Recombinant nucleic acid techniques include, for example, restriction enzyme digestion and ligation, which can be used to isolate a nucleic acid molecule of the invention. In some embodiments, nucleic acids can be obtained using the polymerase chain reaction (PCR). General PCR techniques are described, for example, in PCR Primer: A Laboratory Manual, Dieffenbach & Dveksler, Eds., Cold Spring Harbor Laboratory Press, 1995, and U.S. Pat. No. 4,683,195.

Polypeptides can be expressed from nucleic acids using, for example, expression vectors that contain the appropriate regulatory elements (e.g., transcriptional and/or translational control sequences). See, for example, Sambrook et al., Molecular Cloning: A Laboratory Manual, 2d ed., Cold Spring Harbor Laboratory, New York (1989); and Ausubel et al., Current Protocols in Molecular Biology, Green Publishing Associates and Wiley Interscience, New York (1989). Expression vectors can be used in a variety of cell types (e.g., bacteria, yeast, insect, and mammalian). A wide variety of suitable expression vectors and systems are commercially available, including the pET series of bacterial expression vectors (Novagen, Madison, Wis.), the Adeno-X expression system (Clontech, Mountain View, Calif.), the Baculogold baculovirus expression system (BD Biosciences Pharmingen, San Diego, Calif.), and the pCMV-Tag vectors (Stratagene, La Jolla, Calif.).

The term “isolated” as used herein with reference to nucleic acid refers to a naturally-occurring nucleic acid that is not immediately contiguous with both of the sequences with which it is immediately contiguous (one on the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote host cell.

Any number of different test compounds can be screened in the methods described herein. For example, a compound can be an antibody, an antigen-binding fragment, a small molecule, a synthetic compound, a naturally-occurring compound, a polypeptide, and/or an antigen. For example, there are several small molecule libraries that are commercially available including, for example, ChemBridge (San Diego, Calif.) for small molecule and combinatorial libraries, Enzo Life Sciences (Farmingdale, N.Y.) for compound libraries, and the NIH Molecular Libraries Small Molecule Repository (San Francisco, Calif.). Any such library can be screened to determine whether or not one or more compounds therein bind to a CD20 conformational isomer using the methods described herein. In addition, there are a number of small molecules already identified that bind to cholesterol binding motifs of other membrane proteins (e.g., SKF 10,047 (N-allylnormetazocine), which can be screened for their effects on the conformation of CD20 polypeptide sequences. A compound that exhibits activity in the screening methods described herein (e.g., a candidate compound) can be further evaluated for therapeutic efficacy in the appropriate animal models and clinical trials.

The presence or absence of binding of a test compound to a CD20 polypeptide, wild type or otherwise, can be determined using methods that are routine in the art. For example, compounds that bind to cells or membrane preparations containing CD20 polypeptides can be detected using flow cytometry with fluorescence activated cell sorting (FACS) analysis or variations thereof, immunofluorescence with confocal microscopy, whole cell ELISA using, for example, the standard colorimetric ELISA format or fluorometric microvolume assay technology (FMAT).

Once binding is confirmed, a compound can be evaluated for its ability to cause cell death. Cell death can occur by any number of different mechanisms including, without limitation, cell-killing via, for example, apoptosis, antibody-dependent cellular cytotoxicity (ADCC) or complement dependent cytotoxicity (CDC), inhibiting the translocation of CD20 into lipid rafts, or effecting cell cycle regulation (e.g., causing cell cycle arrest). Methods for evaluating cell death, and cell death via such mechanisms, are well known in the art.

### Antibodies and Methods of Making Antibodies

The extracellular domains of CD20 are extremely small with just two small loops. Therefore, it is perhaps not surprising that almost all CD20 antibodies characterized to date bind essentially the same site in the CD20 sequence, referred to as the “ANPS” (“Ala Asn Pro Ser”) epitope, which is an extracellular epitope that is held in a cyclic conformation and is critical for binding. Ultimately, this may reflect a bias with the immunogen used to generate the CD20 antibodies, which predominantly has been whole B cells (e.g., lymphoma cell lines). Despite the uniformity of the extracellular epitope recognized by the various antibodies (e.g., Rituxumab, B1, and 2H7), CD20 antibodies have been classified into 2 types based on their mechanism of action. Type I anti-CD20 antibodies are those that “open” the CD20 calcium channel, stabilize CD20 into lipid rafts, are highly effective at complement-dependent cytotoxicity (CDC), but are less effective at apoptosis, while type II CD20 antibodies are those that “close” the calcium channel, do not allow stabilization of CD20 into lipid rafts, weakly induce CDC, but are effective at triggering apoptosis. While most of the antibodies produced are classified as type I, both types of CD20 antibodies are effective at antibody-dependent cellular cytotoxicity and both types of antibodies show excellent effectiveness in limiting tumor growth.

Rituxumab is a type I CD20 antibody. In addition to Rituxumab, other FDA-approved CD20 therapeutic antibodies include Ibritumomab and Tositumomab. Also, a number of other second-generation antibodies similar to Rituxumab are in pre-clinical development and clinical trials including, for example, Ofatumumab, Ocrelizumab, TRU-015, Veltuzumab, AME-133v, PRO131921, and GA101, which is a humanized version of Tositumomab. Further, biosimilars to Rituxumab also are undergoing clinical evaluations. Therefore, while some of the disclosure herein refers to Rituxumab, it would be understood by those skilled in the art that any antibody that binds to the extracellular epitope can be used in the screening methods.

In addition to using a known antibody in one or more of the methods described herein, novel antibodies can be made that recognize a CD20 conformational isomer. Methods of making antibodies are well known in the art. As used herein, “antibodies” refer to polyclonal antibodies, monoclonal antibodies, chimeric antibodies, humanized antibodies, and fragments thereof (e.g., a Fab fragment, a Fab′ fragment, or a F(ab′)₂ fragment).

Antibodies that recognize and bind specifically to a conformational isomer of CD20 can be made using a CD20 sequence as described herein. Methods of making antibodies (e.g., polyclonal antibodies) typically include injecting a host animal with an antigenic polypeptide (e.g., a CD20 conformational isomer). Host animals that can be used to produce antibodies include, without limitation, mice, rats, rabbits, goats, sheep, chickens, and horses.

After a host animal has been immunized, the resulting antibody can be collected from the serum (or the egg yolk when chickens are the host animal) and purified using, for example, affinity adsorption to the antigenic polypeptide (e.g., mutant CD20 polypeptide) or a portion thereof. Monoclonal antibodies can be obtained by isolating lymphocytes from the host animal and immortalizing them (e.g., by fusing them to a cancer cell line). Alternatively, monoclonal antibodies can be produced using, for example, recombinant technologies, phage display technologies, synthetic technologies such as CDR-grafting, or combinations of any such technologies.

An antibody produced using such methods binds specifically to a conformational isomer of a CD20 polypeptide and does not recognize or bind to a wild type CD20 polypeptide. In some embodiments, a host animal could be immunized with both a wild type CD20 and a CD20 conformational isomer, in order to develop a pan-specific antibody that binds to both the wild type CD20 and the CD20 conformational isomer.

Methods of Identifying and/or Treating Rituxumab-Resistant Individuals

The identification herein of conformational isomers of CD20 polypeptides, which likely play a role in Rituxumab-resistant B cell cancers and autoimmune diseases, and antibodies produced using such conformational isomers of CD20 polypeptides can be used in a variety of different methods as described herein. For example, the identification of conformational isomers of CD20 polypeptides allows for methods of predicting whether an individual diagnosed with an autoimmune disease will respond well or will respond poorly to treatment with Rituxumab (or a similar therapeutic antibody). As used herein, an “individual” typically refers to a human. An individual can be one that is receiving CD20-antibody therapy, or one that is being screened for their ability to specifically bind Rituxumab or a similar antibody. In addition to humans, “individuals” as used herein can include any other non-human mammal that expresses CD20 or an ortholog thereof. See, for example, FIGS. 7 and 8.

As used herein, B cell lymphomas include, for example, Hodgkin's lymphomas and non-Hodgkin's Lymphomas (NHLs; e.g., diffuse large B cell lymphoma, follicular lymphoma, mucosa-associated lymphatic tissue lymphoma (MALT), small cell lymphocytic lymphoma, and mantle cell lymphoma). In addition, representative autoimmune diseases include, without limitation, rheumatoid arthritis (RA), multiple sclerosis (MS), amyotrophic lateral sclerosis (ALS), celiac disease, Crohns disease, Diabetes mellitus type I, Graves' disease, Guillain-Barre syndrome, Myasthenia gravis, psoriasis and ulcerative colitis.

In some embodiments, a biological sample from an individual can be evaluated for the presence of CD20 polypeptides that bind to an antibody that recognizes and binds to a conformational isomer of CD20. For example, a high level of binding of such an antibody to CD20 polypeptides in the biological sample indicates that the individual will respond poorly to treatment with Rituxumab, while low levels of binding of such an antibody to CD20 polypeptides in the biological sample indicates that the individual will respond well to treatment with Rituxumab.

In some embodiments, an individual can be identified as Rituxumab-resistant or Rituxumab-sensitive using an antibody that recognizes and binds to a conformational isomer of CD20. Typically, an individual is identified as Rituxumab-resistant if an antibody that recognizes and binds to a conformational isomer of CD20 binds a higher level of polypeptides in a biological sample from the individual relative to the level of polypeptides in the biological sample bound by Rituxumab. Alternatively, an individual is identified as Rituxumab-sensitive if Rituxumab binds a higher level of polypeptides in a biological sample from the individual relative to the level of polypeptides in the biological sample bound by an antibody that recognizes and binds to a conformational isomer of CD20.

In addition, an antibody that recognizes and binds to a conformational isomer of CD20 can be used to treat an individual suffering from an autoimmune disease that is not responding or is responding poorly to treatment with Rituxumab. Such an individual may be identified as Rituxumab-resistant and, therefore, may respond better to treatment with an antibody that recognizes and binds to a conformational isomer of CD20. Obviously, the individual can continued to be administered Rituxumab, which still may provide some therapeutic benefit depending on the level of polypeptides that bind to Rituxumab relative to the level that bind to an antibody that recognizes and binds to a conformational isomer of CD20.

Routes of administering compounds including antibodies to an individual are well known in the art and include, for example, parenteral, e.g., intravenous, intradermal, subcutaneous, oral (e.g., ingestion or inhalation), transdermal (topical), transmucosal, and rectal administration. A compound for administering to an individual typically is formulated to be compatible with its intended route of administration. In some cases, a compound can be administered with a pharmaceutically acceptable carrier. The term “pharmaceutically acceptable carrier,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio.

As used herein, “pharmaceutically acceptable carrier” is intended to include any and all solvents, dispersion media, coatings, antibacterial and anti-fungal agents, isotonic and absorption delaying agents, and the like, compatible with the intended route of administration. Pharmaceutical carriers suitable for administration of the compositions provided herein include any such carriers known to those skilled in the art to be suitable for the particular mode of administration. For example, a pharmaceutically acceptable carrier can include a solid, semi-solid, or liquid material that acts as a vehicle, carrier, or medium for a polypeptide. The use of such pharmaceutically acceptable carriers to deliver compounds is well known in the art. Except insofar as any conventional pharmaceutically acceptable carrier is incompatible with the active compound, use thereof in any of the compositions described herein is contemplated. Therapeutic compounds such as antibodies can be formulated using techniques and procedures well known in the art (see, e.g., Ansel, Introduction to Pharmaceutical Dosage Forms, Fourth Edition 1985, 126).

Typically, a compound is administered to an individual such that the individual produces a desired response. An effective amount of a compound is one that reduces or ameliorates at least some of the symptoms associated with a B cell lymphoma or an autoimmune disease in the individual but does not result in significant toxicity. An effective amount of a compound can depend on factors such as, without limitation, the route of administration; the nature of the compound; and the size and weight of the individual. An effective amount of a compound can be established by one of ordinary skill in the art through routine trials establishing dose response curves.

In accordance with the present description, there may be employed conventional molecular biology, microbiology, biochemical, and recombinant DNA techniques within the skill of the art. Such techniques are explained fully in the literature. Representative techniques are described in the following examples, which do not limit the scope of the methods and compositions of matter described in the claims.

### EXAMPLES

### Example 1

### Plasmid Construction

The plasmid pICQ-CC was constructing by gene synthesis of a 723 by DNA fragment containing the CD20 N- and C-terminal cytoplasmic domains attached in frame to the “snorkel tag” (see, for example, U.S. Application No. 61/445,220 filed Feb. 22, 2011). The fragment was digested with EcoRI and AgeI and ligated into the plasmid pCI at the EcoRI and XmaI sites, respectively. The resulting plasmid was designated pICQ-CC.

The dU method was used to clone inserts into the pICQ-CC plasmid. Briefly, the pICQ-CC plasmid was digested with XbaI (TCTAGA) and XmaI (CCCGGG) and two short oligonucleotides were ligated to the 5′ overhang sites generated by the XbaI and XmaI digestion. Inserts were prepared by PCR amplifying the gene with primers that contain a 5′ flanking dU-containing sequence complementary to the oligonucleotides ligated to the pICQ-CC. The PCR products were treated with UDG to create single stranded DNA flanks that could then anneal with the pICQ-CC plasmid, and then were transformed into E. coli to prepare plasmid.

The CD20 gene used was based on the human protein (Uniprot P11826; SEQ ID NO:1) and built from synthetic oligonucleotides using a codon table with frequently used mammalian codons. A kozak sequence (GCCGCCACC (SEQ ID NO:7)) was added to the 5′ end of the gene and universal primer sequences were added to both the 5′ (cac ttc tgg tgc ttc tgg c (SEQ ID NO:8)) and 3′ (aag atc cgc tac ttg ctc c (SEQ ID NO:9)) ends to allow amplification and dU cloning (via XbaI and XmaI sites as discussed above).

The “core” region of CD20, (CD20 minus the N and C terminal domains, region 64-209), was dU cloned into pICQ-CC to create the “CD20+0” mutant (SEQ ID NO:2). The dU cloning process inserts DNA sequences at the 5′ and 3′ ends of the DNA fragment that codes for 8 amino acids and 9 amino acids, respectively (indicated with double-underlining in the sequences below). The CD20+6 (region 58-215; SEQ ID NO:3), CD20+12 (region 52-221; SEQ ID NO:4), and CD20+18 (region 44-227; SEQ ID NO:5) mutants were constructed by gene synthesis and dU cloned into pICQ-CC as described above. They were designated based on the presence of an additional 6, 12 or 18 aa residues, respectively, on both flanks of the core region of CD20. The putative cholesterol binding consensus sequence is indicated with single underlining in the sequences below.

(CD20 wild type)
SEQ ID NO: 1
MTTPRNSVNGTFPAEPMKGPIAMQSGPKPLFRRMSSLVGPTQSFFMRESKTLGAVQIMNGLF
HIALGGLLMIPAGIYAPICVTVWYPLWGGIMYIISGSLLAATEKNSRKCLVKGKMIMNSLSL
FAAISGMILSIMDILNIKISHFLKMESLNFIRAHTPYINIYNCEPANPSEKNSPSTQYCYSI
QSLFLGILSVMLIFAFFQELVIAGIVENEWKRTCSRPKSNIVLLSAEEKKEQTIEIKEEVVG
LTETSSQPKNEEDIEIIPIQEEEEEETETNFPEPPQDQESSPIENDSSP
(CD20+0)
SEQ ID NO: 2
MTTPRNSVNGTFPAEPMKGPIAMQSGPKPLFRRMSSLVGPTQSFFMRESKTLGAVQIMNGLF
HSSTSGASGIALGGLLMIPAGIYAPICVTVWYPLWGGIMYIISGSLLAATEKNSRKCLVKGK
MIMNSLSLFAAISGMILSIMDILNIKISHFLKMESLNFIRAHTPYINIYNCEPANPSEKNSP
STQYCYSIQSLFLGILSVMLIFAFFQELVIAGASSGSSGSGIVENEWKRTCSRPKSNIVLLS
AEEKKEQTIEIKEEVVGLTETSSQPKNEEDIEIIPIQEEEEEETETNFPEPPQDQESSPIEN
DSSP
(CD20+6)
SEQ ID NO: 3
MTTPRNSVNGTFPAEPMKGPIAMQSGPKPLFRRMSSLVGPTQSFFMRESKTLGAVQIMNGLF
HSSTSGASGMNGLFHIALGGLLMIPAGIYAPICVTVWYPLWGGIMYIISGSLLAATEKNSRK
CLVKGKMIMNSLSLFAAISGMILSIMDILNIKISHFLKMESLNFIRAHTPYINIYNCEPANP
SEKNSPSTQYCYSIQSLFLGILSVMLIFAFFQELVIAGIVENEGASSGSSGSGIVENEWKRT
CSRPKSNIVLLSAEEKKEQTIEIKEEVVGLTETSSQPKNEEDIEIIPIQEEEEEETETNFPE
PPQDQESSPIENDSSP
(CD20+12)
SEQ ID NO: 4
MTTPRNSVNGTFPAEPMKGPIAMQSGPKPLFRRMSSLVGPTQSFFMRESKTLGAVQIMNGLF
HSSTSGASGLGAVQIMNGLFHIALGGLLMIPAGIYAPICVTVWYPLWGGIMYIISGSLLAAT
EKNSRKCLVKGKMIMNSLSLFAAISGMILSIMDILNIKISHFLKMESLNFIRAHTPYINIYN
CEPANPSEKNSPSTQYCYSIQSLFLGILSVMLIFAFFQELVIAGIVENEWKRTCSGASSGSS
GSGIVENEWKRTCSRPKSNIVLLSAEEKKEQTIEIKEEVVGLTETSSQPKNEEDIEIIPIQE
EEEEETETNFPEPPQDQESSPIENDSSP
(CD20+18)
SEQ ID NO: 5
MTTPRNSVNGTFPAEPMKGPIAMQSGPKPLFRRMSSLVGPTQSFFMRESKTLGAVQIMNGLF
HSSTSGASGMRESKTLGAVQIMNGLFHIALGGLLMIPAGIYAPICVTVWYPLWGGIMYIISG
SLLAATEKNSRKCLVKGKMIMNSLSLFAAISGMILSIMDILNIKISHFLKMESLNFIRAHTP
YINIYNCEPANPSEKNSPSTQYCYSIQSLFLGILSVMLIFAFFQELVIAGIVENEWKRTCSR
PKSNIGASSGSSGSGIVENEWKRTCSRPKSNIVLLSAEEKKEQTIEIKEEVVGLTETSSQPK
NEEDIEIIPIQEEEEEETETNFPEPPQDQESSPIENDSSP

### Example 2

### Cell Culture

FreeStyle™ 293-F cells were obtained from Invitrogen Corporation. Cells were sub-cultured as outlined by the manufacturer. Briefly, cells were grown in FreeStyle™ 293 Expression Medium (Invitrogen Corporation) in 125 mL shaker flasks. Flasks were seeded at a density of 1×10⁵ viable cells/mL (30 mL final volume). Flasks were incubated in a humidified incubator at 37° C., 8% CO₂ on an orbital shaker platform rotating at 130 rpm. Cell density and viability was monitored and cells were sub-cultured when the density reached 1×10⁶ viable cells/mL.

### Example 3

Transfections

Twenty-four hours before the transfection, the 293-F cells were sub-cultured at a density of about 6×10⁵ cells/mL. The day of transfection, the viability of the cells was determined to be >90% and the cells were diluted to a density of 1×10⁶ cells/mL and 3 mL was placed in each well of a 6 well culture plate. The plasmid DNA was diluted as recommended for the FreeStyle™ 293-F cells.

Briefly, DNA was mixed to OptiPro™ SFM. In a second tube, Invitrogen's FreeStyle™ MAX reagent was added to a total OptiPro™ SFM and mixed. The contents of the two tubes were incubated for 5 mins. Equal volumes of the two tubes were then mixed and incubated for 30 minutes at room temperature. The mixture was added slowly, with swirling, to the flask containing the cells. The flask was incubated at 37° C., 8% CO₂ on an orbital shaking platform rotating at 130 rpm.

### Example 4

### Flow Cytometry

Flow cytometry was performed on a Guava EasyCyte Plus (Millipore, Billerica, Mass.). Briefly, 2-5×10⁴ transfected cells were placed in each well of a 96 well V bottom plate and stained with saturating amounts of fluorescently-labeled (phycoerythrin (PE)) monoclonal antibodies. All staining was in a final volume of 50 μA of 10% normal goat serum (heat inactivated, 30 minutes at 56° C.) in PBS with 0.025% sodium azide and performed at 2-8° C. After 30 minutes with gentle shaking, cells were washed three times with cold 1% bovine serum albumen (BSA) in PBS with 0.025% sodium azide and analyzed. To stain cells with mouse antiserum, cells were incubated for 30 min with antiserum diluted 1:100 in 10% normal goat serum (heat inactivated, 30 minutes at 56° C.) in PBS with 0.025% sodium azide. Cells were washed twice and stained with an anti-mouse PE labeled antibody. Flow cytometer calibration was performed using Rainbow Calibrator Particles (Spherotech, Lake Forest, Ill.).

For surface staining, only viable cells as judged by their light scatter characteristics (forward angle and side scatter) were gated to be included in the analysis. Total staining (surface plus internal) was performed using Fix and Perm Cell Permeabilization Kit (Invitrogen, Camarillo, Calif.) as follows: duplicate wells were stained as described previously. After staining and washing, one well of the replicate was fixed using 50 μl of the kit medium A for 30 minutes at room temperature. After washing, cells were resuspended in 50 μl of kit medium B to permeabilize the cells, and antibody was again added. After staining for 30 minutes at 2-8° C. with gentle shaking, cells were washed with cold 1% BSA in PBS, 0.025% sodium azide and 0.1% saponin to facilitate washing. Cells were then analyzed, along with the replicate that received only the surface staining

### Example 5

### Results

Ion channel chimeras between two ion channels were constructed for the purpose of creating boosting ion channel expression for antibody production. The core transmembrane and extracellular domains of one ion channel were fused to the cytoplasmic N- and C-terminal domains of a well expressed ion channel. A candidate protein to use for these chimeras was CD20 because it is expressed at very high levels. The cloning strategy to create the chimeras relied on the dU method and resulted in the insertion of an 8-9 aa linker inbetween the two chimera partners (FIG. 1). A snorkel tag (red) was added at the C-terminus to allow the surface expression of the construct to be monitored (HA tag).

As a control, the CD20 core, from transmembrane domains 1 to 4 (residues 64 to 209), was reinserted into the pICQ-CC plasmid to recreate CD20, albeit with the dU linker inserted. The site of the insertion of the dU linkers is at the predicted junction inbetween the cytoplasmic and the transmembrane domains at the N- and C-terminal ends. The expression of this construct, CD20+0, was compared to wild type CD20 cloned into the pSNKL-Q plasmid containing an identical snorkel tag. Flow cytometry with a HA antibody to the snorkel tag in transiently transfected HEK293 cells showed that both constructs were expressed at similar levels (FIG. 2). Unexpectedly, when the same cells were stained with a CD20 antibody (R&D Systems, Catalog #396444) to an undefined extracellular epitope, the CD20+0 (SEQ ID NO:2) construct failed to stain at all (FIG. 2).

The same result was observed with the CD20 antibody, 2H7, which binds the Rituxumab epitope “ANPS”, and a panel of other CD20 antibodies (FIG. 3). Notably, the CD20+0 mutant was not recognized by any of the CD20 antibodies used. Significantly, these included CD20 antibodies with diverse properties such as FMC7, which is very sensitive to cholesterol, and B1, which does not trigger migration of CD20 into Triton x100-resistant lipid rafts. Furthermore, staining was not present when MBCD was added, which extracts cholesterol from the cell membranes (FIG. 3).

Intracellular staining of cells did not show evidence of intracellular trapping, and demonstrated that most of the CD20 molecules were on the surface of the cells. To further investigate the disappearance of the extracellular epitope, three additional CD20 constructs were made, in which the junction position for the dU cloning site was shifted away from the transmembrane domains by 6 amino acids (aa) (CD20+6; SEQ ID NO:3), 12 aa (CD20+12; SEQ ID NO:4), and 18 aa (CD20+18; SEQ ID NO:5). Testing these using flow cytometry showed that the CD20+0 (SEQ ID NO:2) and CD20+6 (SEQ ID NO:3) constructs did not have the CD20 epitope, (perhaps partial recovery with CD20+6), but appeared fully restored in the CD20+12 (SEQ ID NO:4) and CD20+18 (SEQ ID NO:5) constructs (FIG. 4). Note, that, since the snorkel tag showed surface expression of all the constructs, this means that the CD20 proteins successfully passed the quality control systems in the secretory pathway (ERAD) and cannot be simply mis-folded and trapped inside, and/or destroyed.

Subsequent studies with a wider panel of CD20 antibodies, 2H7, FMC7, and B1, showed the same pattern, with no epitopes recognized in the CD20+0 (SEQ ID NO:2) and CD20+6 (SEQ ID NO:3) constructs. These antibodies were selected because they represent a range of different CD20 antibodies. Binding of 2H7 is dependent on CD20 assembling as a tetramer, efficient binding of FMC7 requires the presence of cholesterol, and binding of B1 does not cause CD20 to translocate into Triton x100 lipid rafts. Treating cells with MBCD to extract cholesterol, or binding at room temperature versus the usual 4° C., did not restore the epitopes.

A polyclonal mouse antiserum was generated by DNA immunization with full length wild type human CD20 (Uniprot P11826). The CD20 antiserum was used to stain CD20 and the CD20 mutant, CD20+0 (SEQ ID NO:2), expressed in HEK293 cells (FIG. 5). This demonstrated that, while most of the staining disappeared on the CD20+0 mutant relative to the wild type protein (red circle), there was a less abundant epitope that stained both CD20 and CD20+0 (black circle). The antibody binding this common epitope may be useful as a therapeutic reagent that more broadly targets CD20, despite conformational changes. The dominant epitope in the antiserum (red circle) did not stain CD20+6 (SEQ ID NO:3) but did stain CD20+12 (SEQ ID NO:4) and CD20+18 (SEQ ID NO:5), in a manner similar to other CD20 antibodies (e.g., 2H7). Conversely, the less abundant epitope (black circle) stained all of the CD20 mutants (CD20+0 (SEQ ID NO:2), CD20+6 (SEQ ID NO:3), CD20+12 (SEQ ID NO:4), and CD20+18 (SEQ ID NO:5)) as well as wild type CD20 (SEQ ID NO:1; see FIG. 5). The fact that the antiserum stains the cells expressing the mutant CD20 molecules also indicates that the mutant molecules are present on the cells and not eliminated by other causes such as proteolysis.

Inspection of the sequences in CD20 interrupted by the dU linker with the known features and motifs showed that the C-terminal insertion sites partially overlapped with the region known to be important for translocation of CD20 into lipid rafts upon the binding of an antibody to the extracellular domain (e.g., residues 216-226 (SEQ ID NO:10; FIG. 6). Moreover, the mutations that have been identified in clinical samples from patients that exhibit a loss of the CD20 epitope map to this region; 1211S, T219A, and E215G substitutions.

Motif analysis (e.g., SCANPROSITE from expasy.ch/cgi-bin/prosite on the World Wide Web) of these N- and C-terminal membrane juxtaposition regions did not reveal any matches with known motifs. A manual comparison to motifs implicated in lipid raft association showed a possible presence of a soho motif (found in some proteins that associate with lipid rafts via flotillin association; see, e.g., Kimura et al., 2001, PNAS USA, 98:9098), but the identity was weak and not highly convincing.

Since CD20 is influenced by cholesterol, the CD20 sequence was manually scanned for cholesterol binding motifs. A cholesterol binding motif was first identified in the transmembrane protein peripheral-type benzodiazepine receptor (PBR) and subsequently found in several other proteins. The key amino acid residues identified by mutagenesis and conserved evolutionarily resulted in the consensus sequence: LN(X₁₋₅)Y(X₁₋₅)R/K) (Li et al., 1998, Endocrinology, 139:4991). A cholesterol binding consensus sequence was identified in CD20 and the sequence alignment is shown below. The only difference is that the Y is substituted for W, which are similar amino acids in that they are both aromatic. Because the sequence motif is small, it would be expected to occur relatively commonly and, thus, the match may be considered insignificant. In addition, the putative CD20 cholesterol binding site is positioned directly adjacent to a transmembrane domain, which is similar to the cholesterol binding sites identified in other proteins. Note that, in the CD20+0 (SEQ ID NO:2) and CD20+6 (SEQ ID NO:3) mutants, no sequences were deleted. Rather, the C-terminal sequences were simply moved further away from the membrane. Importantly, the putative cholesterol binding motif was disrupted in the CD20+0 (SEQ ID NO:2) and CD20+6 (SEQ ID NO:3) constructs, which showed ablation of the epitope, but not in the CD20+12 (SEQ ID NO:4) and CD20+18 (SEQ ID NO:5) constructs, where the epitope was restored. The cholesterol binding sequence in CD20 is conserved across orthologs in other species (FIG. 7, Table 1).

L/V (X₁₋₅)Y (X₁₋₅)R/K
(SEQ ID NO: 6)
G I V E N E W K R T C S

Some cholesterol binding proteins have more than one motif (e.g., the signal receptor has two) (Palmer et al., 2007, Cancer Res., 67:11166). Searching the other seven juxtamembrane positions in CD20 for the carbohydrate binding motif revealed two additional putative sites in the extracellular domain. These two sites are evolutionary conserved across orthologs (FIG. 8). It is noted that it was assumed that the consensus sequence was reversed (i.e., from (L/V-Y-K/R) to (K/R-Y-L/V)) when it was found N terminal to the membrane, instead of C-terminal as in the original consensus, since it would bind cholesterol in the membrane from a different orientation.

### Example 6

### Additional Mutants

A number of CD20 mutants were constructed to further describe the conformational states and to examine the mechanism of action. Mutants were constructed similarly to the CD20+12 mutant, except the two linkers at the N- and C-terminal regions were tested separately at the +12 position. For example, the linkers were inserted at the +12 position at the N-terminus and +0 position in the C-terminus (CD20N, region 52-209), or vice versa (CD20C, region 64-221), to determine which insertion site(s) disrupt(s) the epitope. It was found that antibody binding was reduced 10- to 20-fold with the CD20N sequence (i.e., disruption of the C-terminal site), and antibody binding was virtually completely abolished with the CD20C sequence (i.e., disruption of the N-terminal site). As described above, the C-terminal site contains the putative cholesterol binding site, whereas the N-terminal site had no identifiable motif. However, analysis of the N-terminal site with transmembrane domain prediction programs (TMPRED, TOPPRED, SOSUI, TMHMM) showed considerable variability in the position of the transmembrane domain with predictions extending up to the +13 position. Since the crystal structure of CD20 has not been determined, and in the absence of other supporting biochemical data, it is difficult to determine the true position of the transmembrane domain. Thus, one possibility is that the CD20C mutant disrupts the extracellular epitope by disturbing the transmembrane domain and, thus, disrupts the folding of the entire protein.

In addition, a series of mutants were constructed as modified forms of the CD20+12 mutant to evaluate the spatial sensitivity of the motif. In these constructs, in addition to the insertions at the +12 positions in the N- and C-terminal flanks, different length peptides (1 aa, 3 aa, and 6 aa inserts) were inserted at the +0 position at the C-terminal flank. Results indicated that the longer peptide insertions correlated with increased perturbation of the extracellular epitope.

Also, mutants are constructed in which the amino acids within the 12 residues adjacent to the membrane in the cholesterol binding motif are mutated. Such mutants include W216A, E213A, V212A, R218A, Y191A and F146A. Further, mutants are constructed in which the extracellular domains of CD20 are mutated in order to map the position of the epitope for any novel antibodies generated.

Any of the CD20 mutant sequences that exhibit reduced or significantly reduced binding by one or more of the extracellular epitope-binding antibodies are used to immunize mice to generate antibodies that bind to CD20 conformational isomers. In addition, the mutants are used to distinguish and classify novel epitopes generated from wild type CD20. Dual staining is used to determine if such antibodies compete with binding for other CD20 antibodies.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A mutant CD20 nucleic acid, wherein said mutant CD20 nucleic acid has a sequence selected from the group consisting of SEQ ID NO:2, SEQ ID NO:3, SEQ ID NO:4, and SEQ ID NO:5.

2. A vector comprising the mutant CD20 nucleic acid of claim 1.

3. A host cell comprising the vector of claim 2.

4. A method of screening for compounds that specifically bind to a conformational isomer of the CD20 polypeptide comprising:
contacting a mutant CD20 polypeptide with a test compound, and
determining whether or not the test compound binds to the mutant CD20 polypeptide,
wherein binding of the test compound to the mutant CD20 polypeptide indicates a compound that binds to a conformational isomer of the CD20 polypeptide.

5. A method of screening for compounds that specifically bind to a conformational isomer of the CD20 polypeptide and to a wild type CD20 polypeptide comprising:
contacting a mutant CD20 polypeptide with a test compound;
contacting a wild type CD20 polypeptide with the test compound; and
determining whether or not the test compound binds to both the mutant CD20 polypeptide and the wild type CD20 polypeptide,
wherein binding of the test compound to both the mutant CD20 polypeptide and the wild type CD20 polypeptide indicates a compound that binds to both a conformational isomer of the CD20 polypeptide and to the wild type CD20 polypeptide.

6. A method of screening for compounds that specifically bind to a conformational isomer of the CD20 polypeptide and induce a conformational change comprising:
contacting a mutant CD20 polypeptide with a test compound in the presence of an extracellular epitope-binding antibody; and
determining whether or not the antibody binds to the mutant CD20 polypeptide in the presence of the test compound,
wherein binding of the antibody to the mutant CD20 polypeptide in the presence of the test compound is indicative of a test compound that induces a conformational change in the conformational isomer of the CD20 polypeptide.

7. The method of claim 4, 5 or 6, wherein the mutant CD20 polypeptide has a sequence selected from the group consisting of SEQ ID NO:2 (CD20+0) and SEQ ID NO:3 (CD20+6).

8. The method of claim 4, 5 or 6, wherein the mutant CD20 polypeptide contains a mutation in the cholesterol binding motif (GIVENEWKRTCS; SEQ ID NO:6).

9. The method of claim 4, 5 or 6, wherein the mutant CD20 polypeptide contains a cholesterol binding motif (GIVENEWKRTCS; SEQ ID NO:6) that is in a different position in the mutant CD20 polypeptide compared to a wild type CD20 polypeptide.

10. The method of claim 4, 5 or 6, wherein the compounds are selected from the group consisting of small molecules, polypeptides, synthetic compounds, naturally-occurring compounds, antibodies, antigen-binding fragments, and antigens.

11. The method of claim 4, 5 or 6, wherein the determining step utilizes FACS analysis.

12. The method of claim 4, 5 or 6, wherein the conformational isomer of the CD20 polypeptide is a CD20 polypeptide that is not specifically bound by an extracellular epitope-binding antibody.

13. A method of making an antibody that specifically binds a conformational isomer of the CD20 polypeptide, comprising the steps of:
immunizing a host animal with a mutant CD20 polypeptide.

14. An antibody directed toward a conformational isomer of a CD20 polypeptide, wherein the antibody is made by the method of claim 13.

15. A method of treating an individual suffering from an autoimmune disease, comprising:
identifying the individual as Rituxumab-resistant or Rituxumab-sensitive, wherein the individual is identified as Rituxumab-resistant if the antibody of claim 14 binds more polypeptides in a biological sample from the individual relative to Rituxumab or wherein the individual is identified as Rituxumab-sensitive if an extracellular epitope-binding antibody binds more polypeptides in a biological sample from the individual relative to the antibody of claim 14; and
administering Rituxumab to the individual if the individual is identified as Rituxumab-sensitive and administering the antibody of claim 14 to the individual if the individual is identified as Rituxumab-resistant.

16. A method of treating an individual who has an autoimmune disease but is not responding or is responding poorly to treatment with Rituxumab, comprising:
administering an effective amount of the antibody of claim 14 to the individual.

17. The method of claim 16, further comprising continuing to administer Rituxumab to the individual.

18. A method of determining whether an individual diagnosed with an autoimmune disease will respond well or will respond poorly to treatment with Rituxumab, comprising:
evaluating the binding of the antibody of claim 14 to CD20 polypeptides in a biological sample from the individual,
wherein a high level of binding of the antibody of claim 14 to CD20 polypeptides in the biological sample indicates that the individual will respond poorly to treatment with Rituxumab, and wherein low levels of binding of the antibody of claim 14 to CD20 polypeptides in the biological sample indicates that the individual will respond well to treatment with Rituxumab.

19. The method of claim 15, 16 or 18, wherein the autoimmune disease is selected from the group consisting of non-Hodgkin's lymphomas (NHL), Hodgkin's lymphoma, and rheumatoid arthritis (RA).


SEQUENCE LISTING (54 sequences)

SEQ ID NO: 1 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 2 - protein, 314 aa, Artificial Sequence
  synthetic peptide
MTTPRNSVNGTFPAEPMKGPIAMQSGPKPLFRRMSSLVGPTQSFFMRESKTLGAVQIMNGLFHSSTSGASGIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGASSGSSGSGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 3 - protein, 326 aa, Artificial Sequence
  synthetic peptide
MTTPRNSVNGTFPAEPMKGPIAMQSGPKPLFRRMSSLVGPTQSFFMRESKTLGAVQIMNGLFHSSTSGASGMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEGASSGSSGSGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 4 - protein, 338 aa, Artificial Sequence
  synthetic peptide
MTTPRNSVNGTFPAEPMKGPIAMQSGPKPLFRRMSSLVGPTQSFFMRESKTLGAVQIMNGLFHSSTSGASGLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSGASSGSSGSGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 5 - protein, 350 aa, Artificial Sequence
  synthetic peptide
MTTPRNSVNGTFPAEPMKGPIAMQSGPKPLFRRMSSLVGPTQSFFMRESKTLGAVQIMNGLFHSSTSGASGMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIGASSGSSGSGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 6 - protein, 12 aa, Homo sapiens
  CD20 cholesterol binding sequence
GIVENEWKRTCS

SEQ ID NO: 7 - DNA, 8 bp, Artificial Sequence
  oligonucleotide
gccgccac

SEQ ID NO: 8 - DNA, 19 bp, Artificial Sequence
  oligonucleotide
cacttctggtgcttctggc

SEQ ID NO: 9 - DNA, 19 bp, Artificial Sequence
  oligonucleotide
aagatccgctacttgctcc

SEQ ID NO: 10 - protein, 24 aa, Homo sapiens
GIVENEWKRTCSRPKSNIVLLSAE

SEQ ID NO: 11 - protein, 12 aa, Homo sapiens
GIVENEWKRTCS

SEQ ID NO: 12 - protein, 12 aa, Musc musculus
GIVENEWKRMCT

SEQ ID NO: 13 - protein, 12 aa, Canis lupus
GIVENEWKKLCS

SEQ ID NO: 14 - protein, 12 aa, Bos taurus
GTVENEWKKLCS

SEQ ID NO: 15 - protein, 12 aa, Meriones unguiculatus
GIVENEWRKMCS

SEQ ID NO: 16 - protein, 12 aa, Ailuropoda melanoleuca
GIVENEWKKLCS

SEQ ID NO: 17 - protein, 12 aa, Equus ferus caballus
GTVENEWKKLCS

SEQ ID NO: 18 - protein, 12 aa, Felis catus
GIVENEWKKLCS

SEQ ID NO: 19 - protein, 12 aa, Oryctolagus cuniculus
GIVENEWKRMCS

SEQ ID NO: 20 - protein, 12 aa, Didelphis virginiana
CITENEWQTLCS

SEQ ID NO: 21 - protein, 12 aa, Ornithorhynchus anatinus
GITENEWRGLCS

SEQ ID NO: 22 - protein, 13 aa, Mus musculus
ATVLNYYVWRDNS

SEQ ID NO: 23 - protein, 13 aa, Rattus norvegicus
ATMLNYYVWRDNS

SEQ ID NO: 24 - protein, 12 aa, Homo sapiens
ATTLNYCVWRDN

SEQ ID NO: 25 - protein, 11 aa, Bos taurus
GMLNYRMWQDN

SEQ ID NO: 26 - protein, 14 aa, Rattus norvegicus
PWVAYHQYYQRPIG

SEQ ID NO: 27 - protein, 14 aa, Homo sapiens
PWVAYHQYYQRPIG

SEQ ID NO: 28 - protein, 14 aa, Bos taurus
PWLAYHRYYGKPIG

SEQ ID NO: 29 - protein, 14 aa, Sus scrofa
PWVAYHZHYQKPVG

SEQ ID NO: 30 - protein, 12 aa, Mus musculus
NPTLNEYHTRAK

SEQ ID NO: 31 - protein, 13 aa, Mus musculus
TFTVTKYWFYRLL

SEQ ID NO: 32 - protein, 14 aa, Homo sapiens
GAKKVFYVIETREP

SEQ ID NO: 33 - protein, 13 aa, Mus musculus
EMLFIYSHFKQAT

SEQ ID NO: 34 - protein, 14 aa, Mus musculus
KLCAGSSYRHMRNG

SEQ ID NO: 35 - protein, 14 aa, Rattus norvegicus
RVYKEMYKTDLEKD

SEQ ID NO: 36 - protein, 13 aa, Rattus norvegicus
QFVLCTYVVSRDP

SEQ ID NO: 37 - protein, 14 aa, Streptococcus spp.
ETWVSLYLAITKNP

SEQ ID NO: 38 - protein, 14 aa, Mus musculus
WVTEGMYAFCYRVM

SEQ ID NO: 39 - protein, 13 aa, Nocardia spp.
ASVTEAYRQRSFA

SEQ ID NO: 40 - protein, 16 aa, Homo sapiens
SLQTLSPYNKGLIRRI

SEQ ID NO: 41 - protein, 14 aa, Oryctolagus cuniculus
STLVVDYIDEGRLV

SEQ ID NO: 42 - protein, 47 aa, Homo sapiens
KISHFLKMESLNFIRAHTPYINIYNCEPANPSEKNSPSTQYCYSIQS

SEQ ID NO: 43 - protein, 47 aa, Canis lupus
TISHFFKMENLNLIKAPMPYVDIHNCDPANPSEKNSLSIQYCGSIRS

SEQ ID NO: 44 - protein, 47 aa, Ailuropoda melanoleuca
TISHFFKMENLNLIKVSTPYINIHNCEPANPAEKNSLSIQYCDSIRS

SEQ ID NO: 45 - protein, 48 aa, Felis catus
AISHFFKMENLNLLKSPKPYIDIHTCQPESKPSEKNSLSIKYCDSIRS

SEQ ID NO: 46 - protein, 47 aa, Equus ferus caballus
TISHFFKWEGLKDDKPSEPYVNIYNCESANPAERNTLSIQYCKSIRS

SEQ ID NO: 47 - protein, 47 aa, Bos taurus
TISHFFKMENLNLIKTQVPYININNCERANPDENNSQSVEYCARIRF

SEQ ID NO: 48 - protein, 47 aa, Oryctolagus cuniculu
TMSHFLKMESLNLIKASTSLINIYNCEPTIPSEKNSPSIQYCYSVQS

SEQ ID NO: 49 - protein, 47 aa, Mus musculus
TLSHFLKMRRLELIQTSKPYVDIYDCEPSNSSEKNSPSTQYCNSIQS

SEQ ID NO: 50 - protein, 47 aa, Didelphis virginiana
TVSHFLKLKSLNLGQTATPYVNIYNCEQNEPSKQSSPFHQYCLCLKS

SEQ ID NO: 51 - protein, 41 aa, Ornithorhynchus anatinus
TISHFFKMESLYLAKTSMPYINIYSCEQSGISQQYCLGMRA

SEQ ID NO: 52 - protein, 8 aa, Artificial Sequence
  synthetic peptide
SSTSGASG

SEQ ID NO: 53 - protein, 9 aa, Artificial Sequence
  synthetic peptide
GASSGSSGS

SEQ ID NO: 54 - protein, 14 aa, Artificial Sequence
  synthetic peptide
SGSQKKPRYEIRWK